(19) United States
(12) Patent Application Publication
(10) Pub. No.: US 2003/0191338 A1
(43) Pub. Date: Oct. 9, 2003

(54) Methods for inhibiting the polymerization of methacrylate monomers

(76) Inventors: Allen D. Johnston, West Chester, PA (US); George F. Cowperthwaite, Coatesville, PA (US)

Correspondence Address: Connolly Bove Lodge & Hutz LLP, P.O. Box 2207, Wilmington, DE, 19899-2207, US

(21) Appl. No.: 10/272,144

(22) Filed: Oct. 16, 2002

Related U.S. Application Data
(60) Provisional application No. 60/370,932, filed on Apr. 9, 2002.

(51) Int. Cl. C07C 69/00

(52) U.S. Cl. 560/4

(57) ABSTRACT

An alpha tocopherol is disclosed as a polymerization inhibitor effect against (meth)acrylate monomer polymerization in nail extension liquids. Alpha-tocopherol was found to inhibit polymerization of ethyl methacrylate.

Priority is claimed under 35 U.S.C. §119(e) from U.S. Provisional Application Serial No. 60/370,932, filed Apr. 9, 2002; the entirety of which is herein incorporated by reference.

### FIELD OF THE INVENTION

The present invention relates to methods for inhibiting the polymerization of methacrylate monomers. More particularly, the present invention relates to a method for inhibiting polymerization of (meth)acrylate monomers during processing and storage of monomer mixtures used in the dental, audiological, medical, and cosmetic fields by using an effective amount of alpha-tocopherol (Vitamin E).

### BACKGROUND OF INVENTION

Polymerizable methacrylate monomers undesirably spontaneously react during various stages of the manufacture, processing, handling, storage and use thereof. It is well known that methacrylate monomers readily polymerize during storage and that such polymerization increases with concurrent temperature increases. Utilization of meth(acrylate) monomers in the dental restorative field involving denture manufacture or in the audiological field of hearing aid shell manufacture requires stability of the monomer mixtures until the operator desires polymerization to ensue. In the field of cosmetic materials, creation of an artificial human nail structure requires that a coating that can be easily applied, polymerizes readily and rapidly due to controlled initiation. U.S. Pat. No. 6,080,389 to Sheariss et al, and U.S. Pat. No. 5,830,442 issued to Beaver describe artificial human nails. To reduce the chance of uncontrolled initiation of polymerization, methacrylate monomers are frequently treated with polymerization inhibitors. Methacrylate monomers are treated with a variety of polymerization inhibitors such as hydroquinone (HQ), methyl ether of hydroquinone (MeHQ), sterically hindered hydrophenols such as 2,6-di-tert-butyl-4-methylphenol (also known as butylated hydroxytoluene, BHT), and sterically hindered quinones such as di-tert-butylhydroquinone.

U.S. Pat. No. 5,461,124 (“'124 patent”) discloses the use of Vitamin E as an inhibitor against premature polymerization in reactive systems capable of undergoing a free radical initiated polymerization which system before and/or after polymerization thereof are placed in tissue contact with a living body. The '124 patent is dealing with methyl methacrylate and poly(methylmethacrylate) of bone cements. The reactive composition of the '124 patent is monocomponent or multicomponent medical and/or dental-medical adhesives, cements, or fillers based on conventional monofunctional and/or polyfunctional olefinically unsaturated compounds, particularly acrylic acid and or methacrylic acids or derivatives thereof.

U.S. Pat. No. 5,159,106 (“'106 patent”) discloses the use of Vitamin E as an inhibitor for the synthesis of methacrylate esters of polyhydric alcohols by reaction of the reactants in the presence of acidic esterification catalysts with addition of polymerization inhibitors. The reactor interior is purged with an oxygen-containing gas stream. The process disclosed in the '106 patent is characterized in that part of the reactor interior filled with the gas phase and charged with finely divided liquid droplets containing polymerization inhibitor.

U.S. Pat. No. 6,106,820 describes cosmetic compositions that include nail polish that can optionally contain vitamins.

### SUMMARY OF THE INVENTION

We have discovered that alpha-tocopherol (Vitamin E) is highly effective against (meth)acrylate monomer polymerization under various conditions. In particular, alpha-tocopherol was found to inhibit polymerization of ethyl methacrylate under accelerated aging conditions that simulate processing, long term storage conditions and use of nail enhancement liquids. An object of the invention was to find a way to inhibit polymerization of ethyl methacrylate under accelerated aging conditions that simulate processing, long term storage conditions and use of nail enhancement liquids.

### DETAILED DESCRIPTION OF THE INVENTION

The present invention relates to a method for inhibiting the polymerization of (meth)acrylate monomer or mixtures of monomers comprising adding an effective inhibiting amount of alpha-tocopherol alone or in part with other inhibitors. The other inhibitors can be, but are not limited to, hydroquinone, methyl ether of hydroquinone, butylated hydroxytoluene, benzoquinone, tert-butyl catechol and other hindered catechols. If the inhibitors are present, the ratio of alpha-tocopherol to the inhibitors is preferably from about 1 to about 9 to about 9 to about 1.

The alpha-tocopherol in nail enhancement liquids is effective at inhibiting the polymerization of (meth)acrylate monomers during conditions encountered in processing, storage, transportation, and use. These conditions employ heat and will often cause premature, uncontrolled and unwanted polymerization of the liquid. The alpha-tocopherol is added to said methacrylate monomer in an amount ranging from about 1 to about 100,000 parts per million of methacrylate and more preferably from about 1 to about 1,000 parts per million of methacrylate.

The (meth)acrylate monomers used in dental, audiological, or cosmetic liquids are generally described as ethylenically unsaturated monomers. A primary component of dental and audiological liquids is methyl methacrylate. The methyl methacrylate is not generally used alone, but is generally mixed with other mono and multifunctional meth(acrylate) monomers. A primary component of nail enhancement liquids is ethyl methacrylate. The ethyl methacrylate is not generally used alone, but it is generally mixed with other components. Again, U.S. Pat. No. 6,080,389 to Sheariss et al, and U.S. Pat. No. 5,830,442 issued to Beaver describe artificial human nails and the ingredients that are used in the manufacture of the nails. The nail enhancement liquids are mixtures of mono- di- and poly functional (meth)acrylate molecules. The ethylenically unsaturated monomer may be mono-, di-, tri or polyfunctional. A variety of ethylenically unsaturated monomers are suitable, so long as the monomers selected are capable of polymerization directly on the nail surface to yield a polymerized artificial nail structure upon exposure to the proper stimuli.

The (meth)acrylate monomers of the present invention are characterized by a CH₂═CR₁—C(O)OR₂ grouping and are highly reactive and polymerize easily. The CH₂═CR₁—C(O)OR₂ grouping is referred to as an ethylenically unsaturated monomer. Examples of a suitable monofunctional ethylenically unsaturated monomer include those where R₁ is H, a C₁₋₃₀ straight or branched chain alkyl, aryl, aralkyl; R₂ is pyrrolidinone, or a substituted or unsubstituted aromatic, alicyclic, or bicyclic ring where the substituents are C₁₋₃₀ straight or branched chain alkyl, or COOM wherein M is H, C₁₋₃₀ straight or branched chain alkyl, pyrrolidinone, or a substituted or unsubstituted aromatic, alicyclic, or bicyclic ring where the substituents are C₁₋₃₀ straight or branched chain alkyl which may be substituted with one or more hydroxyl groups, or [(CH₂)ₘO]ₙH wherein n is 1-20 and m is 1-200.

Preferably, the monofunctional ethylenically unsaturated monomer is one wherein R₁ is H or C₁₋₃₀ alkyl, and R₂ is COOM wherein M is C₁₋₃₀ straight or branched chain alkyl which may be substituted with one or more hydroxyl groups.

More preferably, R₁ is H or CH₃ and R₂ is COOM wherein M is a C₁₋₁₀ straight or branched chain alkyl which may be substituted with one or more hydroxyl groups. In the preferred embodiment of the invention, the alpha-tocopherol inhibits a mixture of monofunctional ethylenically unsaturated monomers wherein R₁ is H or C₁₋₃₀ alkyl, and R₂ is COOM wherein M is C₁₋₃₀ straight or branched chain alkyl and a second monomer wherein R₁ is H or C₁₋₃₀ alkyl, and R₂ is COOM wherein M is C₁₋₃₀ straight or branched chain alkyl which may be substituted with one or more hydroxyl groups. The methacrylate monomer is preferably ethyl methacrylate. The methacrylate monomer comprises at least one methacrylate group of the formula

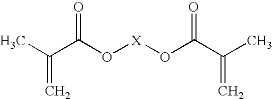

wherein

X isCH₂—CH₂, the ethylene glycol dimethacrylate molecule, CH₂—CH₂—O—CH₂—CH₂ or diethylene glycol dimethacrylate, CH₂—CH₂—O—CH₂—CH₂—O—CH₂—CH₂ or triethylene glycol dimethacrylate, CH₂—CH₂—O—CH₂—CH₂—O—CH₂—CH₂—O—CH₂—CH₂ or tetraethylene glycol dimethacrylate CH₂—CH₂—(O—CH₂—CH₂)ₙ, —O—CH₂—CH₂—, —(CH₂)ₙ—, CH₂—CH₂—C(CH₃)H— or 1,3-butanediol dimethacrylate and

n=3 to 60.

The methacrylate monomer can also be trimethylolpropane trimethacrylate. The meihacrylate monomer can also be of the formula

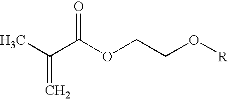

wherein R is H, methyl, ethyl, CH₂—CH₂—O—CH₃, CH₂—CH₂—O—CH₂—CH₃, (CH₂—CH₂—O)ₙ—CH₃ or (CH₂—CH₂—O)ₙ—CH₂—CH₃ wherein n=2 is 60.

The following examples are representative of the invention, but are not intended to limit the invention.

### EXAMPLE 1

Testing was performed to demonstrate the effectiveness of the present invention at inhibiting the polymerization of (meth)acrylate monomers. Tests were conducted in sealed brown glass bottles filled with ethyl methacrylate and various inhibitors. The bottles were placed in a 75° C. water bath in the dark and checked daily for polymer. The monomers remained liquid until they polymerized to a solid.

TABLE 1
Stability Data

| | Days to Polymerization |
|---|---|
| Vitamin E in EMA storage at 75° C. | |
| Vitamin E (ppm) | |
| 102 | 29 |
| 205 | 40 |
| 410 | 54 |
| MeHQ (ppm) | |
| 15 | 15 |
| 95 | 56 |
| BHT (ppm) | |
| 208 | 29 |

MeHQ is the Methyl ether of Hydroquinone and BHT is butylated hydroxytoluene. From this data it can be concluded that all additives tested (Vitamin E, MeHQ and BHT) provide some stability to the Ethyl Methacrylate liquid. The more inhibitor added to the ethyl methacrylate, the longer the liquid maintains stability. Comparatively speaking, the Vitamin E is approximately twice as powerful an inhibitor as BHT and MeHQ is four times a better inhibitor as Vitamin E.

Using the accepted convention that raising the temperature 10° C. doubles the rates of reaction, the above data in comparative form at room temperature yields the following room temperature stability expectations.

TABLE 2
Expected Stability Vitamin E stabilized EMA storage at 25° C.

| | Days to Polymerization |
|---|---|
| Vitamin E (ppm) | |
| 102 | 938 |
| 205 | 1,280 |
| 410 | 1,728 |
| MeHQ (ppm) | |
| 15 | 480 |
| 95 | 1,792 |
| BHT (ppm) | |
| 208 | 928 |

These results indicate that the compound of the present invention is effective at inhibiting polymerization of (meth)acrylate monomers under conditions approximating storage.

### EXAMPLE 2

Testing was performed to determine that the addition of Vitamin E to meth(acrylate) solutions did not disrupt controlled polymerization of a nail system. The standard test for efficiency and efficacy is to determine the time of the peak temperature for controlled polymerizations. These tests are accomplished by rapidly mixing 2 parts polymer containing benzoyl peroxide and 1 part meth(acrylate) liquid and allowing the mass to polymerize while the temperature of the mass is monitored. The time of the peak temperature is correlated with performance in the dental laboratory. For a dental liquid, ethyl methacrylate (EMA) serves as an adequate model. The liquid contains an amine which reacts with benzoyl peroxide (BPO) in the powder to generate radicals which cure the material. Dimethyl-para-toluidine (DMPT) is a commonly used amine in the dental industry. The liquid also contains additives to tint the liquid and absorb UV light to stabilize the polymer. The compounds listed as UV-9 and violet are commonly employed for these purposes. The methyl ether of hydroquinone (MEHQ) is used as the polymerization inhibitor in standard liquids.

Standard Dental Liquid

| Liquid | | | Polymer | | | Cure time |
|---|---|---|---|---|---|---|
| Composition: | Amount | Amount | Composition | Amount | Amount | Minutes |
| EMA | 96% | | Poly(ethyl methacrylate) | | | |
| MEHQ | 75 ppm | | BPO | 1.99% | | |
| DMPT | 2% | | | | | |
| UV-9 | 2% | | | | | |
| Violet | 1 ppm | | | | | |
| Final Mixture | | 2 mL | | | 4 mL | 10.2 +/− 0.4 |
| EMA | 96% | | Poly(ethyl methacrylate) | | | |
| Vitamin E | 200 ppm | | BPO | 1.99% | | |
| DMPT | 2% | | | | | |
| UV-9 | 2% | | | | | |
| Violet | 1 ppm | | | | | |
| Final Mixture | | 2 mL | | | 4 mL | 9.8 +/− 0.4 |

The only difference between these two groups is the inhibitor package. One is inhibited with 75 ppm of MEHQ and the other is inhibited with 200 ppm Vitamin E. The group size for each of these was five samples. That is five separate mixtures of liquid and powder were made and the polymerization time was monitored for the MEHQ stabilized monomer and for the Vitamin E stabilized group. The five times were averaged together and reported above. Using a Student's t-test for significant differences it was found that these two groups are not significantly different. Each cured at the same time. Thus Vitamin E behaves the same as MEHQ in stability of monomers and creation of polymers.

### EXAMPLE 3

Testing was performed to determine that the addition of Vitamin E to meth(acrylate) solutions did not disrupt controlled polymerization of a nail extension system. The standard test for efficiency and efficacy is to determine the time of the peak temperature for controlled polymerizations. These tests are accomplished by rapidly mixing 2 parts (by volume) polymer containing benzoyl peroxide and 1 part (by volume) meth(acrylate) liquid and allowing the mass to polymerize while the temperature of the mass is monitored. The time of the peak temperature is correlated with performance in the nail salon. For a nail liquid, ethyl methacrylate with a cross linker, tetraethyleneglycol dimethacrylate (TEGDMA) serves as an adequate model. The liquid contains an amine which reacts with benzoyl peroxide (BPO) Sin the powder to generate radicals which cure the material. Dimethyl-para-toluidine (DMPT) is a commonly used amine in the nail industry. The liquid also contains additives to absorb UV light to stabilize the polymer (Tinuvin P). The methyl ether of hydroquinone (MEHQ) is used as the polymerization inhibitor in standard liquids

Standard Nail Liquid

| Liquid | | | Polymer | | | Cure time |
|---|---|---|---|---|---|---|
| Composition: | Amount | Amount | Composition | Amount | Amount | Minutes |
| EMA | 93.5% | | Poly(ethyl methacrylate) | | | |
| MEHQ | 75 ppm | | BPO | 1.99% | | |
| DMPT | 1% | | | | | |
| TEGDMA | 5% | | | | | |
| Tinuvin P | 0.5% | | | | | |
| Final Mixture | | 2 mL | | | 4 mL | 11.8 +/− 0.4 |
| EMA | 93.5% | | Poly(ethyl methacrylate) | | | |
| Vitamin E | 200 ppm | | BPO | 1.99% | | |
| DMPT | 1% | | | | | |
| TEGDMA | 5% | | | | | |
| Tinuvin P | 0.5% | | | | | |
| Final Mixture | | 2 mL | | | 4 mL | 11.0 +/− 0.7 |

The only difference between these two groups is the inhibitor package. One is inhibited with 75 ppm of MEHQ and the other is inhibited with 200 ppm Vitamin E. The group size for each of these was five samples. That is five separate mixtures of liquid and powder were made and the polymerization time was monitored for the MEHQ stabilized monomer and for the Vitamin E stabilized group. The five times were averaged together and reported above. Using a Student's t-test for significant differences it was found that these two groups are not significantly different. Each cured at the same time. Thus Vitamin E behaves the same as MEHQ in stability of monomers and creation of polymers.

While this invention has been described with respect to particular embodiments thereof, it is apparent the numerous other forms and modifications of this invention will be obvious to those skilled in the area. The appended claims and this invention generally should be construed to cover all such obvious forms and modifications which are within the true spirit and scope of the present inventions.

All the patents discussed above are incorporated by reference in their entirety for all useful purposes.

What is claimed:

1. A method for inhibiting polymerization of methacrylate monomers during processing, storage, formulation, and transportation which comprises adding to a methacrylate monomer an effective polymerization inhibiting amount of an alpha-tocopherol.

2. The method of claim 1, wherein said alpha-tocopherol is added to said methacrylate monomer in an amount ranging from about 1 to about 100,000 parts per million of methacrylate.

3. The method of claim 1, wherein said alpha-tocopherol is added to said methacrylate monomer in an amount ranging from about 1 to about 1,000 parts per million of methacrylate.

4. The method of claim 1, wherein said methacrylate monomer is ethyl methacrylate or methyl methacrylate.

5. The method of claim 4, wherein said polymerization inhibiting amount of an alpha-tocopherol further comprises the methyl ether of hydroquinone.

6. The method of claim 5, wherein the ratio of alpha-tocopherol to the methyl ether of hydroquinone ranges from about 1 to 9 to about 9 to 1.

7. The method of claim 4, which further comprises hydroquinone and the ratio of alpha-tocopherol to hydroquinone ranges from about 1 to 9 to about 9 to 1.

8. The method of claim 4, which further comprises butylated hydroxytoluene and the ratio of alpha-tocopherol to butylated hydroxytoluene ranges from about 1 to 9 to about 9 to 1.

9. The method of claim 1, wherein said methacrylate monomer is of the formula 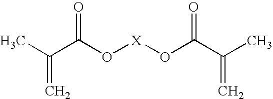
wherein
X is CH₂—CH₂, the ethylene glycol dimethacrylate molecule, CH₂—CH₂—O—CH₂—CH₂ or diethylene glycol dimethacrylate, CH₂—CH₂—O—CH₂—CH₂—O—CH₂—CH₂ or triethylene glycol dimethacrylate, CH₂—CH₂—O—CH₂—CH₂—O—CH₂—CH₂—O—CH₂—CH₂ or tetraethylene glycol dimethacrylate CH₂—CH₂—(O—CH₂—CH₂)ₙ, —O—CH₂—CH₂—, —(CH₂)ₙ—, CH₂—CH₂—C(CH₃)H— or 1,3-butanediol dimethacrylate and
n=3 to 60.

10. The method of claim 1, wherein said methacrylate monomer is trimethylolpropane trimethacrylate.

11. The method of claim 1, wherein said methacrylate monomer is of the formula 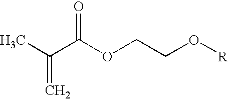
wherein R is H, methyl, ethyl, CH₂—CH₂—O—CH₃, CH₂—CH₂—O—CH₂—CH₃, (CH₂—CH₂—O)ₙ—CH₃ or (CH₂—CH₂—O)ₙ—CH₂—CH₃ wherein n=2 is 60.

12. The method of claim 6, wherein said methacrylate monomer is of the formula 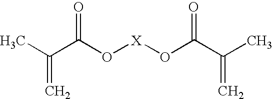
wherein
X isCH₂—CH₂, the ethylene glycol dimethacrylate molecule, CH₂—CH₂—O—CH₂—CH₂ or diethylene glycol dimethacrylate, CH₂—CH₂—O—CH₂—CH₂—O—CH₂—CH₂ or triethylene glycol dimethacrylate, CH₂—CH₂—O—CH₂—CH₂—O—CH₂—CH₂—O—CH₂—CH₂ or tetraethylene glycol dimethacrylate CH₂—CH₂—(O—CH₂—CH₂)ₙ—O—CH₂—CH₂—, —(CH₂)ₙ—, CH₂—CH₂—C(CH₃)H— or 1,3-butanediol dimethacrylate and
n=3 to 60.

13. The method of claim 6, wherein said methacrylate monomer is trimethylolpropane trimethacrylate.

14. The method of claim 6, wherein said methacrylate monomer is of the formula 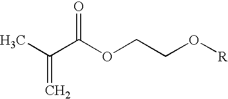
wherein R is H, methyl, ethyl, CH₂—CH₂—O—CH₃, CH₂—CH₂—O—CH₂—CH₃, (CH₂—CH₂—O)ₙ—CH₃ or (CH₂—CH₂—O)ₙ—CH₂—CH₃ wherein n=2 to 60.

15. The method of claim 7, wherein said methacrylate monomer is of the formula 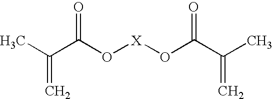
wherein
X isCH₂—CH₂, the ethylene glycol dimethacrylate molecule, CH₂—CH₂—O—CH₂—CH₂ or diethylene glycol dimethacrylate, CH₂—CH₂—O—CH₂—CH₂—O—CH₂—CH₂ or triethylene glycol dimethacrylate, CH₂—CH₂—O—CH₂—CH₂—O—CH₂—CH₂—O—CH₂—CH₂ or tetraethylene glycol dimethacrylate CH₂—CH₂—(O—CH₂—CH₂)ₙ, —O—CH₂—CH₂—, —(CH₂)ₙ—, CH₂—CH₂—C(CH₃)H— or 1,3-butanediol dimethacrylate and
n=3 to 60.

16. The method of claim 7, wherein said methacrylate monomer is trimethylolpropane trimethacrylate.

17. The method of claim 7, wherein said methacrylate monomer is of the formula 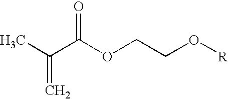
wherein R is H, methyl, ethyl, CH₂—CH₂—O—CH₃, CH₂—CH₂—O—CH₂—CH₃, (CH₂—CH₂—O)ₙ—CH₃ or (CH₂—CH₂—)ₙ—CH₂—CH₃ where n is 2 to 60.

18. The method of claim 1, which further comprises a hindered catechol.

19. The method of claim 1, which further comprises hydroquinone, methyl ether of hydroquinone, butylated hydroxytoluene, benzoquinone, or tert-butyl catechol.

20. The method of claim 1, wherein said methacrylate monomer is
CH₂═CR₁—C(O)OR₂
wherein
R₁ is H, a C₁₋₃₀ straight or branched chain alkyl, aryl or aralkyl;
R₂ is pyrrolidinone, a substituted or unsubstituted aromatic, alicyclic, or bicyclic ring where the substituents are C₁₋₃₀ straight or branched chain alkyl, or COOM wherein M is H, C₁₋₃₀ straight or branched chain alkyl, pyrrolidinone, or a substituted or unsubstituted aromatic, alicyclic, or bicyclic ring where the substituents are C₁₋₃₀ straight or branched chain alkyl which are optionally substituted with one or more hydroxyl groups, or {(CH₂)ₘO}ₙH wherein n is 1-20 and m is 1-200.

21. The method as claimed in claim 20, wherein R₁ is H or C₁₋₃₀ alkyl, and R₂ is COOM wherein M is C₁₋₃₀ straight or branched chain alkyl which is optionally substituted with one or more hydroxyl groups.

22. The method as claimed in claim 20, wherein R₁ is H or CH₃ and R₂ is COOM wherein M is a C₁₋₁₀ straight or branched chain alkyl which is optionally substituted with one or more hydroxyl groups.

23. A dental, audiological, medical, and cosmetic composition which comprises a methacrylate monomer and an effective polymerization inhibiting amount of an alpha-tocopherol.
